(19) United States
(12) Patent Application Publication
(10) Pub. No.: US 2025/0045676 A1
(43) Pub. Date: Feb. 6, 2025

(54) ADAPTIVE WIRELESS SCANNING MANAGEMENT SYSTEM

(71) Applicant: CareFusion 303, Inc., San Diego, CA (US)

(72) Inventors: Brendan John Burgess, San Diego, CA (US); Mustafa Yusufi, San Diego, CA (US); Ramkumar Subramanian, San Diego, CA (US)

(21) Appl. No.: 18/717,231

(22) PCT Filed: Dec. 7, 2022
(86) PCT No.: PCT/US22/81115

§ 371 Date: Jun. 6, 2024

Related U.S. Application Data
(60) Provisional application No. 63/287,438, filed on Dec. 8, 2021.

(51) Int. Cl. G06Q 10/0639; G06Q 10/087; G06Q 10/20; G16H 20/13; G16H 40/20; G16H 40/40

(52) U.S. Cl. CPC G06Q 10/06393; G06Q 10/087; G06Q 10/20; G16H 20/13; G16H 40/20; G16H 40/40

(57) ABSTRACT

A method for improving a performance of scanning data tags in a medication station is provided. The method may include receiving, from a scanner of a medication station, scan data associated with a scan of a plurality of data tags positioned within the medication station. The method may also include generating, based on the scan data, a performance metric indicating a performance of the plurality of data tags. The method may further include adjusting, based on the performance metric, a scan parameter of the scan of the plurality of data tags and/or transmitting, based on the performance metric, an indication associated with the performance of the plurality of data tags. Related methods and articles of manufacture are also disclosed.

### CROSS-REFERENCE TO RELATED APPLICATIONS

The present application claims priority to U.S. Provisional Application No. 63/287,438, filed Dec. 8, 2021, and entitled, “Adaptive Wireless Scanning Management System,” the entirety of which is incorporated by reference herein.

### TECHNICAL FIELD

The subject matter described herein relates to managing systems for wireless scanning, and more particularly, to adaptive systems to scan data tags coupled to items stored in storage devices including wireless scanners such as radio-frequency identifier (RFID) enabled medication dispensing stations.

### BACKGROUND

Institutional pharmacies and medical facilities use medication stations, such as medication dispensing stations, automated dispensing cabinets (“ADCs”), anesthesia stations, and/or the like, to provide convenient access to medication for caregivers in patient care areas. An item, such as a medication or medical supply, is generally stored in unique storage locations, such as drawers of the medication stations, or is otherwise stored within the medication station.

The medication stations may track inventory of the item. For example, medication stations may scan data tags associated with the item during an inventory scan after the medication stations are accessed, and items are added or removed from the medication stations. Such inventory scans are generally performed quickly to update the inventory of the medication stations and to allow for appropriate refilling of the item stored in the medication stations. The results from the inventory scans may be inaccurate due to damaged data tags, improperly placed data tags, degrading or failing data tags, improper positioning of the item within the medication stations, a damaged scanner or antenna, improper configuration of the medication station (e.g., the scanner or antenna), or the like. However, inventory scans are generally unable to detect the inaccuracies, and do not generally provide information regarding the performance of the data tags or scanning elements. Thus, it may be difficult to determine when the inventory of the item is inaccurately measured, to determine when or how to correct the positioning of the data tags or item to allow for better inventory tracking, to predict failure of the data tags, to adaptively adjust a scanning parameter of the medication station, or the like.

### SUMMARY

According to some aspects, a method may include receiving, from a scanner of a medication station, scan data associated with a scan of a plurality of data tags positioned within the medication station. The method may also include generating, based on the scan data, a performance metric indicating a performance of the plurality of data tags. The method may also include adjusting, based on the performance metric, a scan parameter of the scanner of the plurality of data tags and/or transmitting, based on the performance metric, an indication associated with the performance of the plurality of data tags.

In some aspects, the scan parameter includes a selection of the scanner from a plurality of scanners of the medication station to scan at least one data tag of the plurality of data tags.

In some aspects, the scan parameter includes one or more of a length of the scan of the plurality of data tags, a scanning path of the scanner during the scan of the plurality of data tags, a frequency of the scan of the plurality of data tags, and a scanning interval between consecutive scans of the plurality of data tags.

In some aspects, the scan is a deep scan performed at a predetermined time when the medication station is not being accessed and/or the medication station has not been accessed within an amount of time.

In some aspects, the deep scan is performed to one or more of assess the performance of the plurality of data tags and improve the performance of the plurality of data tags.

In some aspects, the adjusting includes dynamically adjusting an inventory scan setting of an inventory scan of the plurality of data tags, the inventory scan determining an inventory of a plurality of medications within the medication station after the medication station is accessed, wherein each of the plurality of data tags are coupled to a corresponding medication of the plurality of medications.

In some aspects, the indication indicates one or more of a failure of at least one data tag of the plurality of data tags, an unacceptable degradation of at least one data tag of the plurality of data tags, use of an unauthorized data tag, improper placement of at least one data tag of the plurality of data tags, and failure of the scanner.

In some aspects, the method further includes comparing the performance metric to a prior performance metric and/or to a baseline performance metric. The method may also include determining the performance metric does not correspond to the prior performance metric and/or the baseline performance metric

In some aspects, the method also includes generating, based on the determination, a prediction of a failure of at least one data tag of the plurality of data tags. The indication includes the prediction.

In some aspects, the method also includes detecting, based on the determination, use of an unauthorized data tag. The method may also include logging, based on the detection, the unauthorized data tag.

In some aspects, the indication includes: a recommendation for one or more of positioning of the plurality of data tags, performing a maintenance check of the plurality of data tags, and adjusting the scan parameter.

In some aspects, the method also includes determining the performance metric fails to correspond to a threshold metric.

In some aspects, the method includes receiving, from the scanner, updated scan data associated with an updated scan of the plurality of data tags. The updated scan may occur after the adjusting and/or the transmitting. The method may also include generating, based on the updated scan data, an updated performance metric indicating an updated performance of the plurality of data tags.

In some aspects, the method also includes adjusting, based on the updated performance metric, an updated scan parameter of the updated scan of the plurality of data tags and/or transmitting, based on the updated performance metric, an updated indication associated with the performance of the plurality of data tags.

In some aspects, the method also includes requesting one or more of a barcode scan of the plurality of data tags, key access to the medication station, and a witness to view a user accessing the medication station.

In some aspects, the scanner is coupled to a drawer of the medication station.

In some aspects, the scanner includes an antenna.

In some aspects, the scanner includes a plurality of antennas.

In some aspects, the medication station includes one or more of a dispensing station and an anesthesia station.

In some aspects, the plurality of scan data includes one or more of a signal strength of the plurality of data tags, a total scans per scanning period, a total number of scanners reading each of the plurality of data tags, and user information associated with a user positioning the plurality of data tags.

In some aspects, the plurality of data tags includes a plurality of RFID tags.

In some aspects, the plurality of data tags are each coupled to a medication stored within the medication station.

In some aspects, the performance metric includes a performance score.

In some aspects, the method includes associating the performance metric with a first medication station. the method may also include comparing the performance metric of the first medication station with a second performance metric associated with a second medication station. The second performance metric may be generated based on scan data associated with a second scan of a second plurality of data tags positioned within the second medication station. The method may also include displaying, via a user interface, information representing the comparing.

In some aspects, the method also includes associating the performance metric with a first medical facility. The method may also include comparing the performance metric of the first medical facility with a second performance metric associated with a second medical facility. The second performance metric may be generated based on scan data associated with a second scan of a second plurality of data tags positioned within a second medication station of the second medical facility. The method may also include displaying, via a user interface, information representing the comparing.

In some aspects, the method also includes displaying, via a user interface, the performance metric.

According to some aspects, a method includes scanning, by a scanner of a medication station, a plurality of data tags coupled to a plurality of medications positioned within the medication station. The method may also include generating, by the scanner and during the scanning, scan data associated with the scanning. The method may also include transmitting, by the scanner, the scan data. The method may also include receiving, by the scanner, a control signal causing an adjustment of the scanning of the plurality of data tags by the scanner.

According to some aspects, a method includes deep scanning a data tag coupled to a medication stored in the medication station. The method may also include generating, during the deep scanning, scan data associated with the scanning. The method may also include generating, based on the scan data, a performance metric indicating a performance of the data tag. The method may also include adjusting, based on the performance metric, a scan parameter of the scanner of the data tag and/or transmitting, based on the performance metric, an indication associated with the performance of the data tag.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the management of medication stations, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a block diagram illustrating a adaptive wireless scanning management system, in accordance with some example implementations;

FIG. 2 depicts a system diagram illustrating an example of an adaptive wireless scanning management system, in accordance with some example implementations;

FIG. 3 schematically depicts an example adaptive wireless scanning management system, in accordance with some example implementations;

FIG. 4 schematically depicts an example adaptive wireless scanning management system, in accordance with some example implementations;

FIG. 5 depicts a flowchart illustrating a process for improving a performance of scanning data tags in a medication station, in accordance with some example implementations;

FIG. 6 depicts a flowchart illustrating a process for scanning data tags in a medication station, in accordance with some example implementations;

FIG. 7 depicts a flowchart illustrating a process for performing a deep scan in a medication station, in accordance with some example implementations; and

FIG. 8 depicts a block diagram illustrating a computing system, in accordance with some example implementations.

When practical, similar reference numbers denote similar structures, features, or elements.

### DETAILED DESCRIPTION

Institutional pharmacies and medical facilities use medication stations, such as medication dispensing stations, ADCs, anesthesia stations, and/or the like, to deliver medications or other items to nurses or other caregivers as they are needed to treat patients. Medications, such as unique medications, are generally stored in unique storage locations, such as drawers of the medication stations, or are otherwise stored within the medication station. The inventory quantities of the items are governed by minimum and maximum values that, among other things, stimulate requests for refilling the medication stations. The inventory quantities of the items can also be used to monitor the use of certain items.

To monitor the inventory quantities of the medications, medication stations may track inventory of the medication. For example, the medication stations may perform an inventory scan to determine an inventory of medications stored in the medication station. The inventory scans may involve quickly scanning data tags associated with the medications after the medication stations are accessed, such as when medications are added to and/or removed from the medication station and/or when the drawers of the medication station are opened and closed. As described herein, data tags may include radio-frequency identification (RFID) tags, “smart” labels, near field communication (NFC) tags, transponders, and/or the like. In the application, features may be described with reference to medications but will also be recognized as applicable to other items such as medical supplies, supplements, food items, medical tools, nutraceuticals, or other items having or associated with a data tag.

While such inventory scans provide quick results, the results from the inventory scans may be inaccurate due at least in part to damaged data tags, improperly placed data tags, degrading or failing data tags, improper positioning of the medication within the medication stations, a damaged scanner or antenna, improper configuration of the medication station (e.g., the scanner or antenna), or the like. In some instances, a type of medication (e.g., liquids, solids, and the like) and/or a material (e.g., metal) of a medication storage container can cause the data tags coupled to the medication to fail, or be difficult to read. Moreover, in some instances, a user may affect the performance and/or readability of the data tags. For example, a user (e.g., a pharmacist, a caregiver, or the like) may damage the data tags during merchandising or normal use, may improperly apply the data tags to the medication, may use an incorrect tag type, may improperly position the medication in the drawers (e.g., may press the medications against one another, crowd the medication, and the like), may improperly configure a scanning parameter of the medication station, or the like. Furthermore, power level variations, antenna failure, degradation of the antenna or data tag, or the like may reduce performance or readability of the data tags. Accordingly, the quick inventory scans may provide inaccurate scanning data.

However, inventory scans are generally unable to detect the inaccuracies, and do not generally provide information regarding the performance of the data tags. Thus, it may be difficult to determine when the inventory of the medication is inaccurately measured, to determine when or how to correct the positioning of the data tags or medication to allow for better inventory tracking, to predict failure of the data tags, to quickly identify misconfigurations, to automatically adjust misconfigurations, or the like.

To improve the accuracy of the scanning of data tags and performance of the medication stations, the adaptive wireless scanning management system consistent with implementations of the current subject matter may perform one or more deep scans (or a combination of deep scans and inventory scans) at the medication station and generate useful information about the performance of the data tags and scanning, based on the deep scans. Deep scans may be performed at a predetermined time when the medication station is not being accessed or the medication station has not been accessed within an amount of time. Generally, the deep scans may be continuously performed in the background and may be slower than inventory scans. The deep scans may additionally or alternatively be characterized by a scan that is performed for a length of time that corresponds to (e.g., is greater than or is equal to) a threshold scan time, such as 6 seconds, 2 to 4 seconds, 4 to 6 seconds, 6 to 10 seconds, 10 to 30 seconds, 30 to 60 seconds, 60 seconds or greater, and/or other ranges therebetween.

The adaptive wireless scanning management system consistent with implementations of the current subject matter may provide drawer-level, medication station-level, or system level (e.g., facility, integrated delivery network, peer groups, etc.) real-time or near real-time analytics based on at least the deep scans of the data tags associated with the medication stored in the medication station. The analytics may be used to generate metrics that, along with the scan data from scanning the data tags, may be displayed via a dashboard. The generated metrics and/or collected data may be used to monitor medication stations across patient care areas, hospitals or other medical facilities, and other care settings. The adaptive wireless scanning management system described herein may also provide the ability to alert low-performing systems, user issues, and drill-down capabilities to aid in the analysis of root causes of the performance issues.

For example, at the drawer level, the system consistent with implementation of the current subject matter may perform inventory scans and deep scans of medication station drawers (e.g., data tagged drawers) to identify weak data tags, data tags that are degrading, weak antennas or scanners, misconfigured medication stations, and/or the like. The system may additionally or alternatively collect drawer statistics and provide data for predictive maintenance of the data tags and/or medication stations. Based on the collected data, the adaptive wireless scanning management system may adjust one or more parameters and/or suggest an adjustment of one or more parameters of the inventory scans and/or the deep scans to improve the readability of the data tags and/or dynamically improve scan performance. In some implementations, responses for a data tag from two or more deep scans may be compared to identify changes in system performance. If the data tag has not changed locations between two scans, but shows a degraded response at the second time, the degradation may indicate one or more of the scanning issues described.

Additionally or alternatively, at the station level, the adaptive wireless scanning management system may collect use data associated with use of the medication station, such as a user who accessed the station, a user type of the user, the drawers of the medical station that were accessed during use of the medication station, workflows performed during use of the medical station, timestamps indicating the time of use of the medication station, or the like. The adaptive wireless scanning management system may display and/or compare the use data alone, or in combination with the scan data to provide additional insights into the performance of the medication station for improving performance of the medication station. Additionally or alternatively, at the server level, scan data and/or use data from multiple medication stations and/or multiple medical facilities can be compared. The data from the multiple medication stations and/or multiple medical facilities can be used to identify trends and/or trigger alerts to determine systemic issues with data tags, drawers, medication stations, and/or the like. For example, at the item level, if the data tag of the item has changed locations between the two scans, the responses should be relatively similar. However, if one response was substantially weaker than another response at a different location, it may indicate an issue with the device at the weaker response location. Thus, the adaptive wireless scanning management system consistent with implementations can provide useful insight into the performance of the medication station at micro and macro levels, leading to improved performance of the medication stations and improved user experience using the medication stations.

In some implementations, the adaptive wireless scanning management system helps to proactively identify performance issues with the medication stations. For example, based on the scan data collected during scans, such as deep scans, the medication management system may adjust one or more scan parameters of the medication station (e.g., a scanner of the medication station), transmit an indication associated with the performance of the medication stations, and/or display the scan data or comparison of scan data. Thus, the medication management system can help with developing preventative maintenance models for replacing components, such as the data tags, before they fail, predicting failure of the data tags, and suggesting maintenance on the medication stations. Such configurations can help to reduce unplanned downtime during which the medication stations are unusable and to improve the user experience.

Additionally or alternatively, the adaptive wireless scanning management system provides corrective benefits. For example, based on the scan data collected during scans, such as deep scans, the medication management system may adjust one or more scan parameters of the medication station (e.g., a scanner of the medication station), transmit an indication associated with the performance of the medication stations, and/or display the scan data or comparison of scan data. The adjustment, indication, and/or displayed data can be used to correct individual user actions, such as improving the placement or method of placement of the data tags. Such configurations may also be used to transmit alerts when medication stations or certain data tags require immediate attention. Furthermore, the insights may trigger automatic adjustments to the wireless scanning system such as configuring the wireless scanner of a medication station, the data tag encoder, a labeling device that places data tags on items, or other element of the system. Accordingly, the adaptive wireless scanning management system may improve the user experience by quickly identifying and addressing issues with the performance of the system.

Thus, the adaptive wireless scanning management system may provide a broad level of useful analytics that help to improve performance of medication stations. The adaptive wireless scanning management system may additionally or alternatively proactively predict required maintenance, suggest updated scan settings, and/or adjust scan settings to reduce maintenance or unplanned downtime of the medication stations. Additionally or alternatively, the adaptive wireless scanning management system may correct individual user actions, improve user practices, and/or quickly alert when stations are failing to adequately perform. Additionally or alternatively, the adaptive wireless scanning management system may generate and display a visualization of one or more performance metrics to allow for and/or suggest more efficient medical workflows.

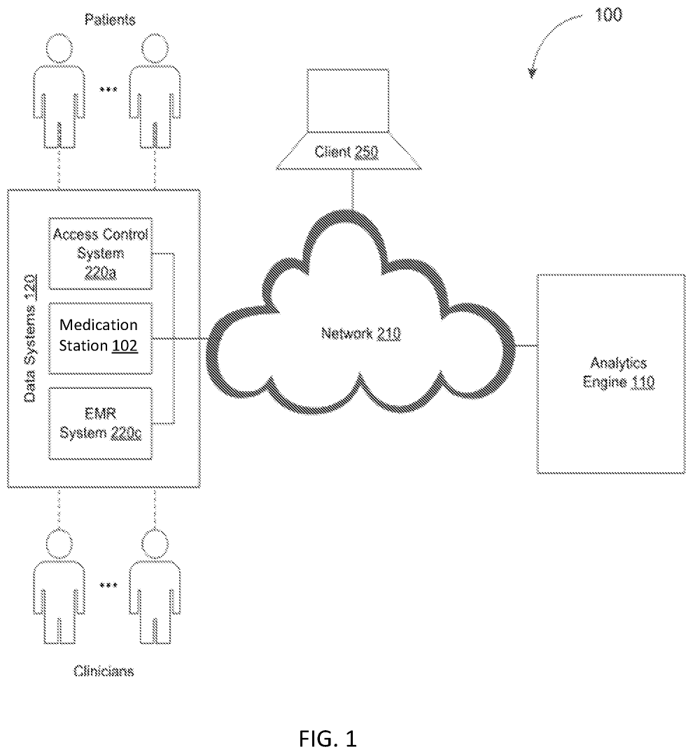

FIG. 1 depicts a system diagram illustrating an example of an adaptive wireless scanning management system 100, consistent with implementations of the current subject matter. As shown in FIG. 1, the adaptive wireless scanning management system 100 may include one or more data systems 120, a client 250, and an analytics engine 110.

The analytics engine 110, the client 250, and the one or more data systems 120 may be communicatively coupled via a network 210. The client 250 may be a processor-based device such as, for example, a smartphone, a tablet computer, a wearable apparatus, a desktop computer, a laptop computer, a workstation, and/or the like. The client 250, which may display one or more generated performance metrics and/or visualizations, may form a part of or be separately coupled to the one or more data systems, such as the medication station 102. The network 210 may be any wired and/or wireless network including, for example, a public land mobile network (PLMN), a local area network (LAN), a virtual local area network (VLAN), a wide area network (WAN), the Internet, and/or the like.

Referring to FIG. 1, the one or more data systems 120 may include an access control system 220a, a medication station 102, an electronic medical record (EMR) system 220c infusion pumps, data tag encoder 122, item-labeling device 124, wasting stations, and/or the like. The medication station 102 may include medication dispensing stations, ADCs, anesthesia stations, and/or the like, to store medications and deliver the medications as they are needed to treat patients. The medication station 102 may be separately coupled to and/or include the analytics engine 110. In some implementations, the analytics engine 110 includes a processor and memory for storing instructions, which when executed by the processor, performs one or more operations described herein.

In some implementations, the medications stored within the medication station 102 may be associated with a data tag. The data tag may be an RFID tag, NFC tag, transponder, or the like. In some implementations, the data tag may be encoded with information by the data tag encoder 122. The system may dynamically configure the data tag encoder 122 to adjust how the data tags are encoded. The adjustment may be based on the scan information collected and processed as described herein. Adjustments may include encoding signal strength, encoding signal time, encoding signal verification process, or the like.

The data tag can be coupled to a corresponding item. In some implementations, the data tag is coupled to a single item. In other implementations, the data tag is coupled to an item container, a group of items, or the like. The data tag may be manually coupled to the item and/or positioned by a user. The adaptive wireless scanning management system 100 may help to improve user's positioning of the data tags on the medication and/or positioning of the medication within the medication station 102. In some implementations, the data tag may be automatically applied to an item by the item-labeling device 124. The item-labeling device 124 may be configured to identify the item and place the data tag on a predetermined location of the item.

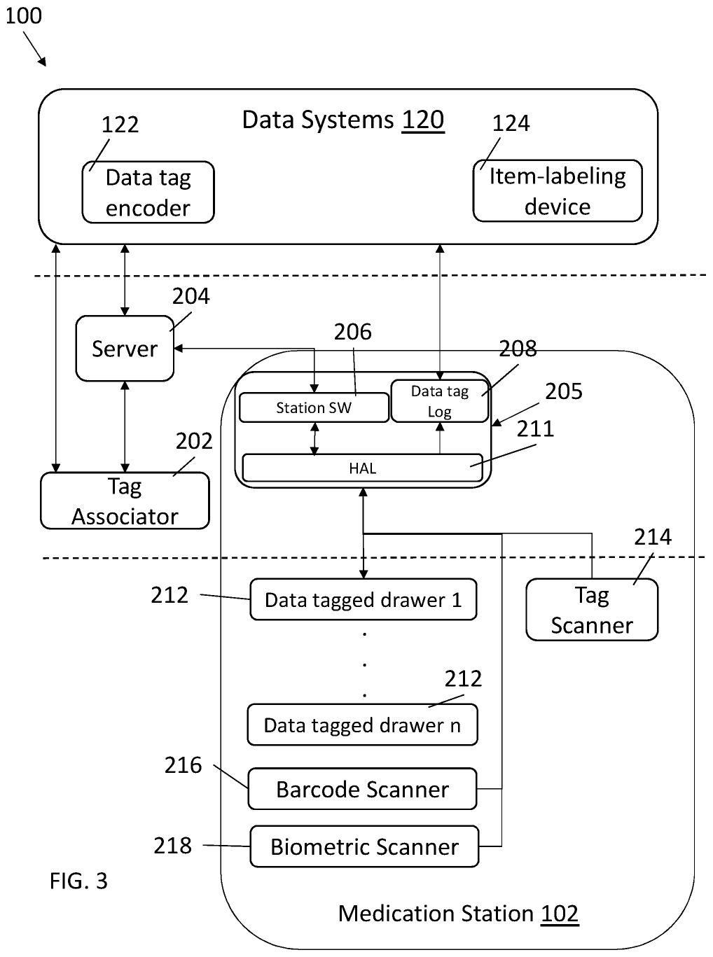

The data tag may be associated with a corresponding medication by a tag associator 202 (see FIG. 3). For example, the tag associator 202 may link the data tag with the corresponding medication. The tag associator 202 may collect data associated with the linking of the data tag and the corresponding medication. For example, the tag associator 202 may collect association data, such as user information (e.g., the user profile, user name, user type, etc.) of the user performing the association, a baseline signal strength of the data tag being applied to the medication or that is already coupled to the medication, the medication type, a packaging type of the item of the item being linked, timestamps of the association, or the like. The tag associator 202 may transmit the association data to the one or more data systems 120 and/or to the analytics engine 110 for further processing and/or for display via a user interface of the client 250. The tag associator 202 may be coupled to and/or form a part of the medication station 102.

Referring to FIG. 3, which schematically depicts an example of the adaptive wireless scanning management system 100, consistent with implementations of the current subject matter, the medication station 102 may include one or more drawers 212, such as data tagged drawers, bins, hangers, and/or the like, a tag scanner 214, a barcode scanner 216, a biometric scanner 218, and/or a station computing system 205. The station computing system 205 may include a station software 206, a hardware abstraction layer 211, and an data tag log 208.

The tag scanner 214 may scan at least one data tag (e.g., a plurality of data tags), such as the data tags positioned within the medication station 102 and associated with the medication. The tag scanner 214 may also be referred to herein as a “scanning device. The tag scanner 214 may include one or more (e.g., one, two, three, four, five to 10, 10 to 15, 15 to 20 or more) scanners. The tag scanner 214 may be positioned at various locations about the medication station 102, such as in the drawers of the medication station 102.

The tag scanner 214 may include an antenna for reading the plurality of data tags and/or transmitting scan data read from the plurality of data tags. For example, the tag scanner 214 may perform one or more scans, such as inventory scans and/or deep scans.

An “inventory scan” refers to a scan of the data tags associated with the medication performed after the medication station 102 is accessed, such as within a period of time (e.g., within one second, two seconds, three seconds, four seconds, five seconds, ten seconds, other ranges therebetween or greater) after the medication station 102 is accessed. The medication station 102 may be considered to be accessed when medications are removed and/or added to the medication station and/or when the drawers or other storage areas within the medication station 102 are opened and closed. Additionally or alternatively, the “inventory scan” is a scan of the data tags associated with the medication that has a scan length that is less than or equal to a threshold scan length (e.g., one second, two seconds, three seconds, four seconds, five seconds, six seconds, seven seconds, eight seconds, nine seconds, ten seconds, other ranges therebetween or lesser). Thus, the inventory scan is performed quickly to determine an inventory of the medication stored within the medication station or within a portion of the medication station.

A “deep scan” refers to a scan of the data tags associated with the medication performed when the medication station 102 is not being accessed, such as at predetermined time intervals, at a predetermined time (e.g., 6 seconds, 2 to 4 seconds, 4 to 6 seconds, 6 to 10 seconds, 10 to 30 seconds, 30 to 60 seconds, 60 seconds or greater, and/or other ranges therebetween) after the inventory scan, and/or at a predetermined time (e.g., 6 seconds, 2 to 4 seconds, 4 to 6 seconds, 6 to 10 seconds, 10 to 30 seconds, 30 to 60 seconds, 60 seconds or greater, and/or other ranges therebetween) after the medication station 102 is accessed. In other words, the deep scans can be performed according to a periodic schedule. Additionally or alternatively, the “deep scan” is a scan of the data tags associated with the item that has a scan length that is greater than or equal to a threshold scan length (e.g., 6 seconds, 2 to 4 seconds, 4 to 6 seconds, 6 to 10 seconds, 10 to 30 seconds, 30 to 60 seconds, 60 seconds or greater, and/or other ranges therebetween). Thus, the deep scans are performed slowly to determine a performance of the data tags which can also be used to identify performance of other elements of the system.

The scans may be performed by the medication station (e.g., a scanner, such as the tag scanners 214, of the medication station) based on one or more scan parameters. The scan parameters may include a selection of a scanner (or group of scanners) from the tag scanners 214 to perform the scanning, a length of scan of the data tags, a scanning path of the tag scanner 214 during the scan of the data tags, a frequency of the scan of the data tags, a scanning interval between consecutive scans, and/or the like. The scan parameters may be dynamically adjusted based on the performance information described.

During the scans, such as the inventory scans and/or the deep scans, the tag scanner 214 may collect scan data. For example, the tag scanner 214 may read the scan data from the data tags at least one (e.g., 1, 5, 100, 500, 1000, 10000, or more) time during the length of the scan. In some implementations, the tag scanner 214 may read the scan data from the data tags a greater number of times during the deep scan than during the inventory scan. As described in more detail below, the analytics engine 110 may determine a mean, median, maximum, and/or minimum of the scan data from each of the data tags. The scan data can be stored at the medication station 102, such as at the data tag log 208. Additionally or alternatively, the scan data can be transmitted by the station computing system 205 to a server (which may be coupled to and/or form a part of the data systems 120) and/or directly to one or more of the data systems 120.

The scan data collected by the tag scanner 214 may include a signal strength of the data tags, a total quantity of scans per scanning period, a total number of scanners reading a particular data tag, user information associated with the user of the medication station and/or the user that positioned the data tags, or the like. In some implementations, user data may also be generated at the medication station 102 when the medication station 102 is accessed. For example, a user, such as a clinician, a caregiver, a pharmacist, a pharmacy courier, or another medical professional interacting with the one or more data systems 120 may trigger the generation of user data. The user data may include a type of user (e.g., a clinician, a caregiver, a pharmacist, a pharmacy courier, and/or another medical service provider), a time stamp associated with the accessing of the medication station 102, the action performed at the medication station 102, an identifier of the user, a device identifier of the medication station 102, a patient identifier of a patient prescribed the medication, an identifier of the medication retrieved from the medication station 102, a quantity of the medication retrieved from the medication station 102, or the like. The user data may include the association data. The user data may be transmitted by the station computing system 205 to a server (which may be coupled to and/or form a part of the data systems 120) and/or directly to one or more of the data systems 120.

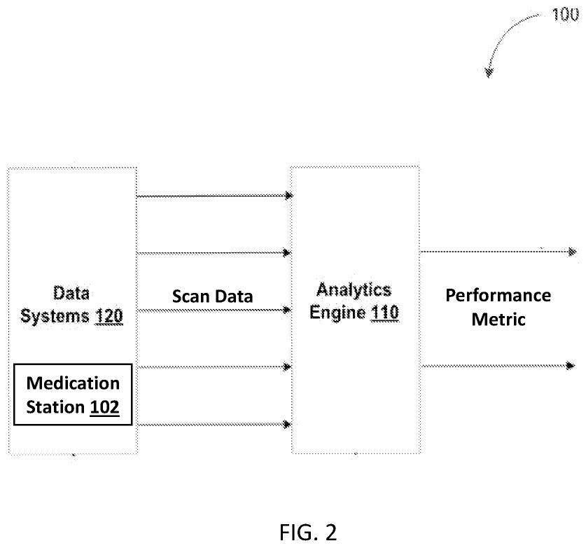

FIG. 2 schematically depicts a system diagram illustrating the adaptive wireless scanning management system 100, in accordance with some example implementations. Referring to FIG. 2, the analytics engine 110 may be communicatively coupled with the one or more data systems 120. The analytics engine 110 may receive, from the one or more data systems 120, a plurality of input data including the scan data, the user data, and/or the association data. Based on the input data, the analytics engine 110 may generate a performance metric indicating a performance of the data tags. The performance metric may include a score, a classifier (e.g., good signal, good performance, weak signal, poor performance, etc.), or the like.

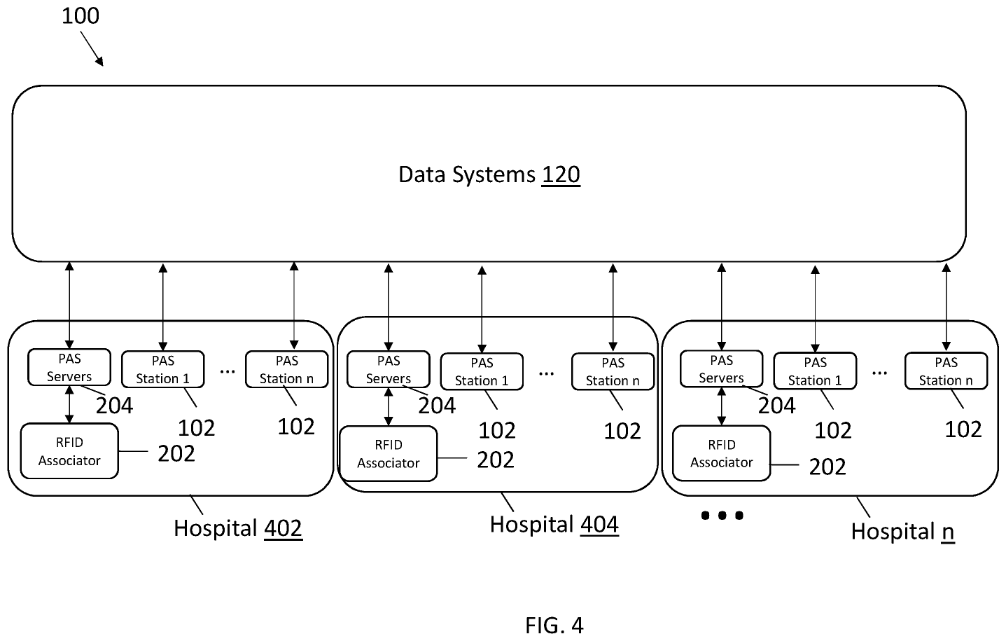

For example, the analytics engine 110 may classify the performance of the data tags by comparing the performance metric to a threshold metric. The performance of the data tags may be determined for each individual data tag, for a group of tags (e.g., positioned within particular drawers and/or within a particular medication station), between different medication stations, between different medical facilities, and/or the like. FIG. 4 depicts a system diagram of the adaptive wireless scanning management system 100, including hospitals 402, 404, n, where each of the hospitals include medication stations 102, servers 204, tag associators 202, data tag encoders 122, item-labeling devices 124, or the like.

As an example, the analytics engine 110 may associate the performance metric with a first medication station. The analytics engine 110 may compare the performance metric to a second performance metric associated with a second medication station. The second performance metric may be generated based on scan data associated with a second scan of a second plurality of data tags positioned within the second medication station. The analytics engine 110 may also display, via a user interface (of the client 250), information representing the comparison, such as in a visualization.

As another example, the analytics engine 110 may associate the performance metric with a first medical facility (e.g., a first hospital). The analytics engine 110 may compare the performance metric to a second performance metric associated with a second medical facility. The second performance metric may be generated based on scan data associated with a second scan of a second plurality of data tags positioned within the second medical facility. The analytics engine 110 may also display, via a user interface (of the client 250), information representing the comparison, such as in a visualization.

Referring back to FIG. 2, the analytics engine 110 may classify the data tags as having strong performance when the scan data indicates the signal strength of the data tags corresponds (e.g., is greater than or equal to) a threshold signal strength, when the total tag readings per scanning period corresponds to a threshold, when the total number of scanners reading a particular data tag corresponds to a threshold, and/or the like. With respect to the total tag readings per scanning period, as described herein, the tag scanner 214 may attempt to read a particular data tag a number of times during the length of the scan. When the analytics engine 110 determines tag scanner 214 reads the particular data tag a number of times that corresponds to a threshold number (e.g., 5, 10, 20, 30, 40, 50, 100, 1000, or the like), the analytics engine 110 classifies the performance of the particular data tag as strong. With respect to the total number of scanners reading a particular data tag, as described herein, the medication station 102 may include a plurality of tag scanners 214. When the analytics engine 110 determines a number of tag scanners 214 that read a particular data tag corresponds to a threshold number (e.g., one, five, ten, fifteen), such that all of the tag scanners 214 or all of the expected tag scanners 214 read the data tag during the scan, the analytics engine 110 classifies the performance of the particular data tag as strong.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

The thresholds described may be static, dynamic, or adaptive. Static thresholds are predetermined thresholds that remain constant. Dynamic thresholds are changed throughout operation of the system based on changes to the system or items stored therein. For example, as the number of items stored increases, it may be desirable to adjust a detection threshold or scanning time or antenna scan sequence. A dynamic threshold may be based on the size of the storage device or antenna arrangement of the storage device. A dynamic threshold may be based on time or date. For example, there may be times when there are fewer people and devices active within an environment. In such instances, the resources needed to achieve a clear scan may be less than the resources needed during a higher traffic period. Adaptive thresholds may be changed in response to changes in characteristics of the storage device and/or items stored therein and may vary depending parameters detectable or received by the system. An adaptive threshold may also be changed in response to detecting degradation of data tags or scanners or misconfiguration of either. Whether static, dynamic, or adaptive, the threshold may be specified as a value or a range of values.

Alternatively, the analytics engine 110 may classify the data tags as having weak performance when the scan data indicates the signal strength of the data tags does not correspond to (e.g., is less than) a threshold signal strength, when the total tag readings per scanning period does not correspond to a threshold, when the total number of scanners reading a particular data tag does not correspond to a threshold, and/or the like. With respect to the total tag readings per scanning period, as described herein, the tag scanner 214 may attempt to read a particular data tag a number of times during the length of the scan. When the analytics engine 110 determines tag scanner 214 reads the particular data tag a number of times that does not correspond to a threshold number (e.g., 5, 10, 20, 30, 40, 50, 100, 1000, or the like), the analytics engine 110 classifies the performance of the particular data tag as weak. With respect to the total number of scanners reading a particular data tag, as described herein, the medication station 102 may include a plurality of tag scanners 214. When the analytics engine 110 determines a number of tag scanners 214 that read a particular data tag does not correspond to a threshold number (e.g., one, five, ten, fifteen), such that only a subset of the tag scanners 214 read the data tag during the scan, the analytics engine 110 classifies the performance of the particular data tag as weak. While the performance is described as weak, the performance may similarly be classified as failing, degrading, or the like.

Based on the input data (e.g., the scan data, the user data, the association data, or the like), the analytics engine 110 may generate or cause display, such as via the client 250, of one or more visualizations. The visualizations may display the input data and/or may indicate the performance of the data tags. For example, the visualizations may display the performance metric. The visualizations may illustrate the performance metric over time, which provides useful information to track and/or update medical workflows performed at a medical facility. The visualizations may additionally or alternatively indicate the performance of a particular data tag, performance of data tags within a particular drawer of a medication station, performance of a particular medication station, performance of a medical facility, and/or the like.

Based on the classification (e.g., weak performance, strong performance, and/or the like), the analytics engine 110 may additionally or alternatively adjust at least one scan parameter of the medication station (e.g., a scanner of the medication station). In other words, the analytics engine 110 may send a control signal to the medication station 102 to adjust operation of the tag scanner 214. The adjustment may improve one or more of the frequency diversity, the spatial diversity, or the channel diversity of the scan. For example, the analytics engine 110 may adjust one or more scan parameters such as a selection of a scanner (or group of scanners) from the tag scanners 214 to perform the scan, a length of scan of the data tags, a scanning path of the tag scanner 214 during the scan of the data tags, a frequency of the scan of the data tags, a scanning interval between consecutive scans, or the like. Such implementations of the current subject matter may improve scanning efficiency and accuracy, and may help to provide improved user experience.

For example, based on the input data from a deep scan, the analytics engine 110 may determine that only one or a subset of the tag scanners 214 are needed to accurately capture the performance of a particular data tag or subset of data tags. As a result, the analytics engine 110 can cause only the necessary tag scanners 214 to scan the selected data tags during a scan, reducing computing resources and improving scanning efficiency. Similarly, based on the input data from the deep scan, the analytics engine 110 may determine an optimal scanning path for future deep scans and/or inventory scans, a minimal length of scan to accurately determine data tag performance, a minimum or maximum scanning interval to improve scanning performance, and/or the like. This allows the analytics engine 110 to dynamically adjust and improve the scanning (e.g., deep scanning and/or inventory scanning) performed by the tag scanners 214.

In some implementations, the analytics engine 110 dynamically causes an adjustment to one or more elements of the system based on the performance of data tags. For example, the analytics engine 110 may transmit a control signal to adjust a scan parameter of a scanning device based on the performance of the data tags detected during another scan. For example, the analytics engine 110 may dynamically adjust a scan parameter of a scanning device during an inventory scan of the data tags. The adjustment may be based on data (e.g., the scan data, user data, device data, encoder data, and/or association data) associated with a deep scan. Thus, the analytics engine 110 can leverage the data received from the deep scans to improve the accuracy and/or efficiency of the inventory scans.

In some implementations, the analytics engine 110 allows for at least one scan parameter to be adjusted via the client 250. For example, the visualization displayed at the client 250 may allow selection of a particular scan parameter and/or adjustment of a scan parameter, and, after selection, adjust the scan parameter. For example, the visualization may present a listing of the tag scanners 214 and/or the data tags. The client 250 may receive a selection of a particular data tag to view the performance of the data tag. Additionally or alternatively, the client 250 may receive a selection of a particular tag scanner 214 or subset of tag scanners 214 to scan each data tag.

Additionally or alternatively, based on the performance metric and/or the input data, the analytics engine 110 transmits an indication associated with the performance of the data tags. The indication may include an alert, such as an audio, visual, audiovisual, and/or tactile alert. The alert may be displayed at the client 250. The indication, such as the alert, may indicate one or more of a failure of at least one data tag, an unacceptable level of degradation of at least one data tag, improper placement of at least one data tag, failure of at least one tag scanner 214, and/or the like. Additionally or alternatively, the indication may include a recommendation for one or more of a proper positioning of the data tags, a recommendation for a proper positioning of the medication, a recommendation for performing a maintenance check of the data tags, a recommendation for an adjustment to at least one scan parameter, or the like.

In some implementations, the indication may indicate use of an unauthorized data tag. For example, if a tag scanner 214 is unable to read a signal from a particular data tag, the analytics engine 110 may determine that the data tag is an unauthorized data tag. Additionally or alternatively, the analytics engine 110 may determine the data tag is an unauthorized data tag when a signal from the data tag does not correspond to a prior signal from the same data tag. An unauthorized data tag may include a third party data tag or another data tag that was not authorized for use with the medication station 102. Upon determining use of the unauthorized data tag, the analytics engine 110 may log the unauthorized data tag, including data associated with the unauthorized data tag such as an identifier, a user who used the data tag, and/or the like.

In some implementations, the indication includes a prediction of a failure of at least one data tag. The prediction of the failure of at least one data tag may cause the medication station 102 storing the medication or item associated with the data tag to be rendered inaccessible until maintenance is performed, may result in maintenance, and/or the like.

In some implementations, the analytics engine 110 may compare the input data and/or the performance metric to a prior scan metric and/or to a baseline scan metric. If the analytics engine 110 determines the performance metric does not correspond (e.g., does not match) the prior scan metric and/or the baseline scan metric, the analytics engine 110 may generate a prediction of failure of at least one data tag and/or may detect that the data tag is unauthorized. The indication may include the prediction and/or data associated with the unauthorized data tag.

In some implementations, the analytics engine 110 may assist with tuning the scanning of the data tags within the medication station. For example, after adjusting at least one scan parameter of the medication station (e.g., a scanner of the medication station), the analytics engine 110 may obtain updated input data (e.g., updated scan data, etc.) associated with an updated scan of the data tags. The updated scan of the data tags may occur after the adjustment to the scan parameter, after the transmission of the indication, after generation of the performance metric, and/or after the collection of initial scan data. The analytics engine 110 may, based on the updated input data, generate an update scan metric indicated an updated performance of the data tags.

Based on the determined updated performance of the data tags, the analytics engine 110 may further adjust the updated scan parameter and/or transmit an updated indication associated with the performance of the data tags. Additionally or alternatively, the analytics engine 110 may determine that at least one tag scanner 214 is not functioning properly or may determine that it is necessary to confirm whether the at least one tag scanner 214 is functioning properly. For example, the analytics engine 110 may determine to bypass the scanning of the data tags by the tag scanner 214. Instead, the analytics engine 110 may request the barcode scanner 216 to scan a barcode of the medication associated with the data tags. Additionally or alternatively, the analytics engine 110 may require key access to the medication station 102 upon determining that the updated performance metric is weak (such as after an adjustment to the scan parameter and/or after a previous weak performance metric). Additionally or alternatively, the analytics engine 110 may require a witness to view a user accessing the medication station 102 upon determining that the updated performance metric is weak (such as after an adjustment to the scan parameter and/or after a previous weak performance metric). Such configurations help to minimize downtime of the medication station 102, while allowing for secure use of the medication station 102 and access to the medications stored within the medication station 102.

The adjustments may be determined based on a performance metric for one or more devices of the system. For example, to adjust a medication station 102, the data tag scan information may be analyzed using artificial intelligence or other operations whereby the system processes inputs and generates outputs with apparent intelligence. The artificial intelligence may be implemented in whole or in part by a model. A model may be implemented as a machine learning model. The learning may be supervised, unsupervised, reinforced, or a hybrid learning whereby multiple learning techniques are employed to generate the model. The learning may be performed as part of training. Training the model may include obtaining a set of training data and adjusting characteristics of the model to obtain a desired model output. For example, three characteristics may be associated with a medication station scanning status. In such instance, the training may include receiving the three characteristics as inputs to the model and adjusting the characteristics of the model such that for each set of three characteristics, the output device state matches the desired scanning status associated with the historical data. In this way, the model can output a value associated with the status of the medication station.

In some implementations, the training may be dynamic. For example, the system may update the model using a set of events. The detectable properties from the events may be used to adjust the model.

The model may be an equation, artificial neural network, recurrent neural network, convolutional neural network, decision tree, or other machine-readable artificial intelligence structure. The characteristics of the structure available for adjusting during training may vary based on the model selected. For example, if a neural network is the selected model, characteristics may include input elements, network layers, node density, node activation thresholds, weights between nodes, input or output value weights, or the like. If the model is implemented as an equation (e.g., regression), the characteristics may include weights for the input parameters, thresholds or limits for evaluating an output value, or criterion for selecting from a set of equations.

Once a model is trained, retraining may be included to refine or update the model to reflect additional data or specific operational conditions. The retraining may be based on one or more signals detected by a device described herein or as part of a method described herein. For example, a deep scan threshold may be defined whereby after the threshold number of deep scans are performed, sufficient data is available to retrain the model. In some implementations, the retraining may be periodic, based on an elapsed time since the model was previously trained. Upon detection of the designated signals, the system may activate a training process to adjust the model as described.

Further examples of machine learning and modeling features which may be included in the embodiments discussed above are described in “A survey of machine learning for big data processing” by Qiu et al. in EURASIP Journal on Advances in Signal Processing (2016) which is hereby incorporated by reference in its entirety.

For analyzing the scan data over a period of time, a recurrent neural network (RNN) model may be trained. The RNN model may be trained using historical scan data for a device or data tag over a period of time along with an indicator of the performance level of the device or data tag over the period of time. This model can then detect the trajectory of a device or data tag based on the sequence or trend of scan data. The model may receive, as an input, the scan data, user data, device data, tag data (e.g., data tag type, encoder used for the data tag, location on an item, etc.), storage location of the item, or the like. The RNN model may then provide, as an output, the expected status of the device or data tag. In some implementations, the model may provide multiple outputs indicating likely status for one or more future time periods.

In some implementations, the need for or adjustment amount may be generated based on configured to compare a slope of the performance metric over time. If the slope of the metric corresponds to a threshold, the system (or other specific element of the system associated with the performance metric) may be degrading or misconfigured. An adjustment may be generated using the slope and/or deviation from the threshold. For example, the slope may be used to adjust the scan time with the expectation that a longer scan time for degrading scanning devices may improve the accuracy. The amount to extend the scan time may be adjusted based on the slope.

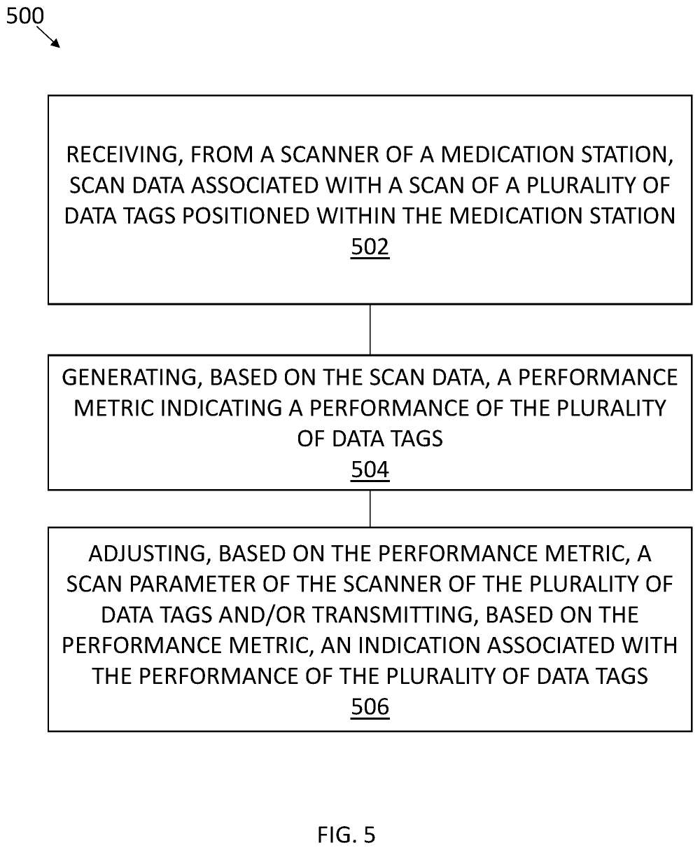

FIG. 5 depicts a flowchart illustrating a process 500 for improving a performance of a medication station, in accordance with some example implementations. Referring to FIG. 5, the process 500 may be performed at least in part by the analytics engine 110.

At 502, the analytics engine 110 may receive scan data from a scanner (e.g., the tag scanner 214) of a medication station (e.g., the medication station 102). The scan data may be associated with a scan of a plurality of data tags positioned within the medication station. The plurality of data tags may each be associated with a medication stored within one or more drawers of the medication station. The scan may include an inventory scan and/or a deep scan, consistent with implementations of the current subject matter. The scan data read by the scanner may include one or more of a signal strength of the plurality of data tags, a total scans per scanning period, a total number of scanners reading each of the plurality of data tags, and user information associated with a user positioning the plurality of data tags, among other data.

At 504, the analytics engine 110 may generate a performance metric based on the scan data. The performance metric may indicate a performance of the plurality of data tags. The performance metric may include a score, a classifier (e.g., good signal, good performance, weak signal, poor performance, etc.), and/or the like.

At 506, the analytics engine 110 may adjust a scan parameter of the medication station (e.g., the scanner of the medication station) of the plurality of data tags based on the performance metric and/or transmit an indication associated with the performance of the plurality of data tags, based on the performance metric.

For example, the analytics engine 110 may adjust one or more of a selection of the scanner from a plurality of scanners of the medication station to scan at least one data tag of the plurality of data tags, a length of the scan of the plurality of data tags, a scanning path of the scanner during the scan of the plurality of data tags, a frequency of the scan of the plurality of data tags, and a scanning interval between consecutive scans of the plurality of data tags. The indication may indicate one or more of a failure of at least one data tag of the plurality of data tags, an unacceptable degradation of at least one data tag of the plurality of data tags, use of an unauthorized data tag, improper placement of at least one data tag of the plurality of data tags, and failure of the scanner. The indication may additionally or alternatively include a recommendation for one or more of positioning of the plurality of data tags, performing a maintenance check of the plurality of data tags, and adjusting the scan parameter.

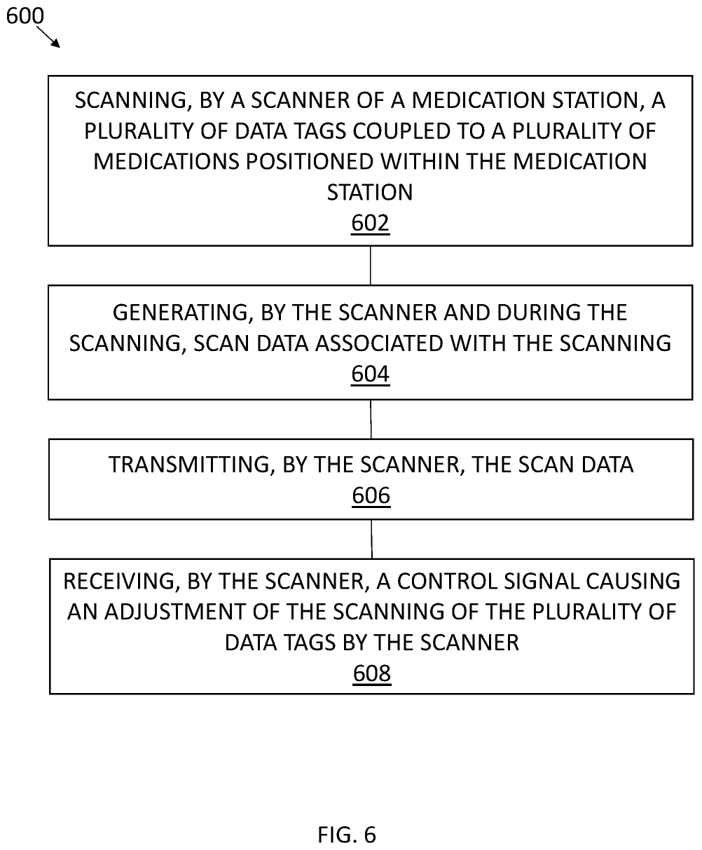

FIG. 6 depicts a flowchart illustrating a process 600 for scanning data tags in a medication station, in accordance with some example implementations. Referring to FIG. 6, the process 600 may be performed at least in part by the medication station 102, such as the tag scanner 214.

At 602, the scanner may scan a plurality of data tags coupled to a plurality of medications positioned within the medication station. The scanner may include a plurality of scanners positioned on and/or coupled to the medication station, such as within at least one drawer of the medication station. The scanner may include an antenna, a transponder, and/or the like, for reading the plurality of data tags. The scanner may be used to perform an inventory scan and/or a deep scan.

At 604, the scanner may generate scan data associated with the scanning of the plurality of data tags. The scan data may include one or more of a signal strength of the plurality of data tags, a total scans per scanning period, a total number of scanners reading each of the plurality of data tags, and user information associated with a user positioning the plurality of data tags, among other data.

At 606, the scanner may transmit the scan data. For example, the scanner may transmit the scan data to the analytics engine 110. Additionally or alternatively, the scanner may transmit the scan data to one or more data systems 120 for later comparison and/or to the client 250 for display as part of a visualization. The scan data alone or in aggregate with other scan data may be processed as described such as using artificial intelligence or other automated analytics to provide performance metrics, adjustments to the system, or the like.

At 608, the scanner may receive a control signal, such as from the analytics engine 110. The control signal may cause an adjustment of the scanning of the plurality of data tags by the scanner. For example, the adjustment may include an adjustment to one or more scan parameters of the medication station (e.g., a scanner of the medication station). The scan parameters may include one or more of a selection of the scanner from a plurality of scanners of the medication station to scan at least one data tag of the plurality of data tags, a length of the scan of the plurality of data tags, a scanning path of the scanner during the scan of the plurality of data tags, a frequency of the scan of the plurality of data tags, and a scanning interval between consecutive scans of the plurality of data tags. The adjustment in the scanning may improve the accuracy and/or efficiency of the scan.

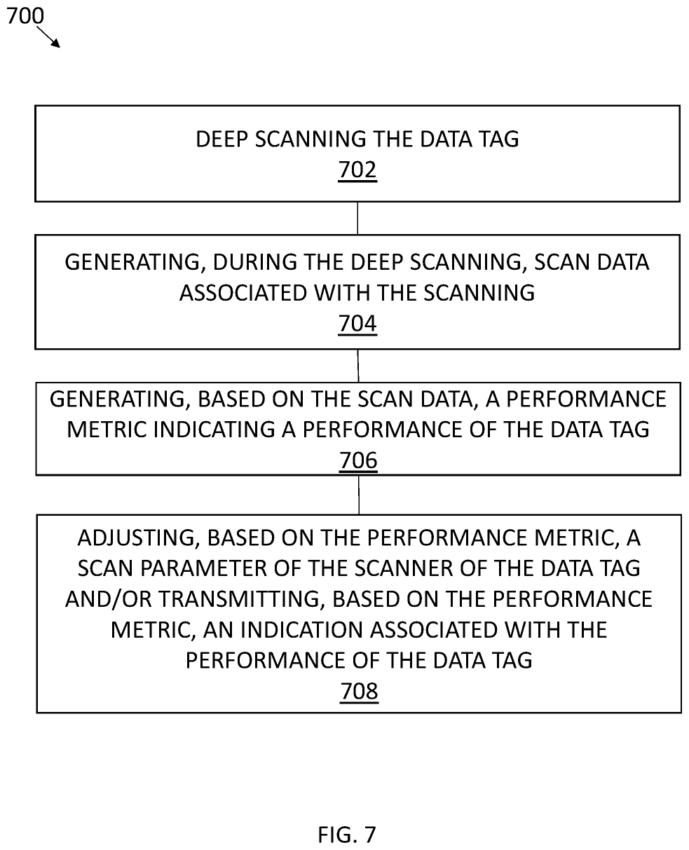

FIG. 7 depicts a flowchart illustrating a process 700 for performing a deep scan in a medication station, in accordance with some example implementations. Referring to FIG. 7, the process 700 may be performed at least in part by the medication station 102, such as by the analytics engine 110 and/or the tag scanner 214 of the medication station 102.

At 702, the medication station 102 may deep scan the data tag. The medication station 102 may perform the deep scan as part of a regular scanning routine, such as in the background. Additionally or alternatively, the medication station 102 may perform the deep scan at regular intervals, after an inventory scan is performed, and/or the like.

At 704, the medication station 102 may generate scan data associated with the scan during the deep scan. The scan data may include one or more of a signal strength of the plurality of data tags, a total scans per scanning period, a total number of scanners reading each of the plurality of data tags, and user information associated with a user positioning the plurality of data tags, among other data.

At 706, the medication station 102 may generate a performance metric based on the scan data. The performance metric may indicate a performance of the plurality of data tags. The performance metric may include a score, a classifier (e.g., good signal, good performance, weak signal, poor performance, etc.), and/or the like.

At 708, the medication station 102 may adjust a scan parameter of the scan of the plurality of data tags based on the performance metric and/or transmit an indication associated with the performance of the plurality of data tags, based on the performance metric.

For example, the medication station 102 may adjust one or more of a selection of a scanner from a plurality of scanners of the medication station to scan at least one data tag of the plurality of data tags, a length of the scan of the plurality of data tags, a scanning path of the scanner during the scan of the plurality of data tags, a frequency of the scan of the plurality of data tags, and a scanning interval between consecutive scans of the plurality of data tags. The indication may indicate one or more of a failure of at least one data tag of the plurality of data tags, an unacceptable degradation of at least one data tag of the plurality of data tags, use of an unauthorized data tag, improper placement of at least one data tag of the plurality of data tags, and failure of the scanner. The indication may additionally or alternatively include a recommendation for one or more of positioning of the plurality of data tags, performing a maintenance check of the plurality of data tags, and adjusting the scan parameter. The adjustments may change the wireless scanning frequency diversity, spatial diversity, or channel diversity. In some implementations, the adjustment may be to a scanning pattern used by a device. For example, some medication stations may include 8 or 16 different antennae. The sequence in which the antennae are activated may impact the quality of the resulting scan data. For example, there may be an antenna that consistently does not perform well. In such instances, the system may provide a scan sequence that prioritizes the antennae to ensure the higher functioning antenna is used before poorer performing antennae. In some cases, the scan sequence may omit an antenna that is performing poorly. In this way, the medication station can conserve resources that would otherwise be spent on activating the poor performing antenna and processing any results therefrom.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:

Example 1: A system, including: at least one data processor; and at least one memory storing instructions which, when executed by the at least one data processor, result in operations including: receiving, from a scanner of a medication station, scan data associated with a scan of a plurality of data tags positioned within the medication station; generating, based on the scan data, a performance metric indicating a performance of the plurality of data tags; and adjusting, based on the performance metric, a scan parameter of the scanner of the plurality of data tags and/or transmitting, based on the performance metric, an indication associated with the performance of the plurality of data tags.

Example 2: The system of example 1, wherein the scan parameter includes a selection of the scanner from a plurality of scanners of the medication station to scan at least one data tag of the plurality of data tags.

Example 3: The system of any one of examples 1 to 2, wherein the scan parameter includes one or more of a length of the scan of the plurality of data tags, a scanning path of the scanner during the scan of the plurality of data tags, a frequency of the scan of the plurality of data tags, and a scanning interval between consecutive scans of the plurality of data tags.

Example 4: The system of any one of examples 1 to 3, wherein the scan is a deep scan performed at a predetermined time when the medication station is not being accessed and/or the medication station has not been accessed within an amount of time.

Example 5: The system of any one of examples 1 to 4, wherein the deep scan is performed to one or more of assess the performance of the plurality of data tags and improve the performance of the plurality of data tags.

Example 6: The system of any one of examples 1 to 5, wherein the adjusting includes dynamically adjusting an inventory scan setting of an inventory scan of the plurality of data tags, the inventory scan determining an inventory of a plurality of medications within the medication station after the medication station is accessed, wherein each of the plurality of data tags are coupled to a corresponding medication of the plurality of medications.

Example 7: The system of any one of examples 1 to 6, wherein the indication indicates one or more of a failure of at least one data tag of the plurality of data tags, an unacceptable degradation of at least one data tag of the plurality of data tags, use of an unauthorized data tag, improper placement of at least one data tag of the plurality of data tags, and failure of the scanner.

Example 8: The system of any one of examples 1 to 7, wherein the operations further include: comparing the performance metric to a prior performance metric and/or to a baseline performance metric; and determining the performance metric does not correspond to the prior performance metric and/or the baseline performance metric.

Example 9: The system of any one of examples 1 to 8, wherein the operations further include: generating, based on the determination, a prediction of a failure of at least one data tag of the plurality of data tags; and wherein the indication includes the prediction.

Example 10: The system of any one of examples 1 to 9, wherein the operations further include: detecting, based on the determination, use of an unauthorized data tag; and logging, based on the detection, the unauthorized data tag.

Example 11: The system of any one of examples 1 to 10, wherein the indication includes: a recommendation for one or more of positioning of the plurality of data tags, performing a maintenance check of the plurality of data tags, and adjusting the scan parameter.

Example 12: The system of any one of examples 1 to 11, wherein the operations further include: determining the performance metric fails to correspond to a threshold metric.

Example 13: The system of any one of examples 1 to 12, wherein the operations further include: receiving, from the scanner, updated scan data associated with an updated scan of the plurality of data tags, the updated scan occurring after the adjusting and/or the transmitting; and generating, based on the updated scan data, an updated performance metric indicating an updated performance of the plurality of data tags.

Example 14: The system of any one of examples 1 to 13, wherein the operations further include: adjusting, based on the updated performance metric, an updated scan parameter of the updated scan of the plurality of data tags and/or transmitting, based on the updated performance metric, an updated indication associated with the performance of the plurality of data tags.

Example 15: The system of any one of examples 1 to 14, wherein the operations further include: requesting one or more of a barcode scan of the plurality of data tags, key access to the medication station, and a witness to view a user accessing the medication station.

Example 16: The system of any one of examples 1 to 15, wherein the scanner is coupled to a drawer of the medication station.

Example 17: The system of any one of examples 1 to 16, wherein the scanner includes an antenna.

Example 18: The system of any one of examples 1 to 17, wherein the scanner includes a plurality of antennas.

Example 19: The system of any one of examples 1 to 18, wherein the medication station includes one or more of a dispensing station and an anesthesia station.

Example 20: The system of any one of examples 1 to 19, wherein the plurality of scan data includes one or more of a signal strength of the plurality of data tags, a total scans per scanning period, a total number of scanners reading each of the plurality of data tags, and user information associated with a user positioning the plurality of data tags.

Example 21: The system of any one of examples 1 to 20, wherein the plurality of data tags includes a plurality of RFID tags.

Example 22: The system of any one of examples 1 to 21, wherein the plurality of data tags are each coupled to a medication stored within the medication station.

Example 23: The system of any one of examples 1 to 22, wherein the performance metric includes a performance score.

Example 24: The system of any one of examples 1 to 23, wherein the operations further include: associating the performance metric with a first medication station; comparing the performance metric of the first medication station with a second performance metric associated with a second medication station, the second performance metric generated based on scan data associated with a second scan of a second plurality of data tags positioned within the second medication station; and displaying, via a user interface, information representing the comparing.

Example 25: The system of any one of examples 1 to 24, wherein the operations further include: associating the performance metric with a first medical facility; comparing the performance metric of the first medical facility with a second performance metric associated with a second medical facility, the second performance metric generated based on scan data associated with a second scan of a second plurality of data tags positioned within a second medication station of the second medical facility; and displaying, via a user interface, information representing the comparing.

Example 26: The system of any one of examples 1 to 25, wherein the operations further include: displaying, via a user interface, the performance metric.

Example 27: A system, including: at least one data processor; and at least one memory storing instructions which, when executed by the at least one data processor, result in operations including: scanning, by a scanner of a medication station, a plurality of data tags coupled to a plurality of medications positioned within the medication station; generating, by the scanner and during the scanning, scan data associated with the scanning; transmitting, by the scanner, the scan data; and receiving, by the scanner, a control signal causing an adjustment of the scanning of the plurality of data tags by the scanner.

Example 28: A medication station, including: at least one data processor; and at least one memory storing instructions which, when executed by the at least one data processor, result in operations including: deep scanning a data tag coupled to a medication stored in the medication station, wherein the medication station includes a scanner configured to scan the data tag; generating, during the deep scanning, scan data associated with the scanning; generating, based on the scan data, a performance metric indicating a performance of the data tag; and adjusting, based on the performance metric, a scan parameter of the scanner of the data tag and/or transmitting, based on the performance metric, an indication associated with the performance of the data tag.

Example 29: The medication station of example 28, further including: the medication; the data tag coupled to the medication; and a scanner configured to scan the data tag.

Example 30: A method, including: receiving, from a scanner of a medication station, scan data associated with a scan of a plurality of data tags positioned within the medication station; generating, based on the scan data, a performance metric indicating a performance of the plurality of data tags; and adjusting, based on the performance metric, a scan parameter of the scanner of the plurality of data tags and/or transmitting, based on the performance metric, an indication associated with the performance of the plurality of data tags.

Example 31: The method of example 30, wherein the scan parameter includes a selection of the scanner from a plurality of scanners of the medication station to scan at least one data tag of the plurality of data tags.

Example 32: The method of any one of examples 30 to 31, wherein the scan parameter includes one or more of a length of the scan of the plurality of data tags, a scanning path of the scanner during the scan of the plurality of data tags, a frequency of the scan of the plurality of data tags, and a scanning interval between consecutive scans of the plurality of data tags.

Example 33: The method of any one of examples 30 to 32, wherein the scan is a deep scan performed at a predetermined time when the medication station is not being accessed and/or the medication station has not been accessed within an amount of time.

Example 34: The method of any one of examples 30 to 33, wherein the deep scan is performed to one or more of assess the performance of the plurality of data tags and improve the performance of the plurality of data tags.

Example 35: The method of any one of examples 30 to 34, wherein the adjusting includes dynamically adjusting an inventory scan setting of an inventory scan of the plurality of data tags, the inventory scan determining an inventory of a plurality of medications within the medication station after the medication station is accessed, wherein each of the plurality of data tags are coupled to a corresponding medication of the plurality of medications.

Example 36: The method of any one of examples 30 to 35, wherein the indication indicates one or more of a failure of at least one data tag of the plurality of data tags, an unacceptable degradation of at least one data tag of the plurality of data tags, use of an unauthorized data tag, improper placement of at least one data tag of the plurality of data tags, and failure of the scanner.

Example 37: The method of any one of examples 30 to 36, further including: comparing the performance metric to a prior performance metric and/or to a baseline performance metric; and determining the performance metric does not correspond to the prior performance metric and/or the baseline performance metric

Example 38: The method of any one of examples 30 to 37, further including: generating, based on the determination, a prediction of a failure of at least one data tag of the plurality of data tags; and wherein the indication includes the prediction.

Example 39: The method of any one of examples 30 to 38, further including: detecting, based on the determination, use of an unauthorized data tag; and logging, based on the detection, the unauthorized data tag.

Example 40: The method of any one of examples 30 to 39, wherein the indication includes: a recommendation for one or more of positioning of the plurality of data tags, performing a maintenance check of the plurality of data tags, and adjusting the scan parameter.

Example 41: The method of any one of examples 30 to 40, further including: determining the performance metric fails to correspond to a threshold metric.

Example 42: The method of any one of examples 30 to 41, further including: receiving, from the scanner, updated scan data associated with an updated scan of the plurality of data tags, the updated scan occurring after the adjusting and/or the transmitting; and generating, based on the updated scan data, an updated performance metric indicating an updated performance of the plurality of data tags.

Example 43: The method of any one of examples 30 to 42, further including: adjusting, based on the updated performance metric, an updated scan parameter of the updated scan of the plurality of data tags and/or transmitting, based on the updated performance metric, an updated indication associated with the performance of the plurality of data tags.

Example 44: The method of any one of examples 30 to 43, further including: requesting one or more of a barcode scan of the plurality of data tags, key access to the medication station, and a witness to view a user accessing the medication station.

Example 45: The method of any one of examples 30 to 44, wherein the scanner is coupled to a drawer of the medication station.

Example 46: The method of any one of examples 30 to 45, wherein the scanner includes an antenna.

Example 47: The method of any one of examples 30 to 46, wherein the scanner includes a plurality of antennas.

Example 48: The method of any one of examples 30 to 47, wherein the medication station includes one or more of a dispensing station and an anesthesia station.

Example 49: The method of any one of examples 30 to 48, wherein the plurality of scan data includes one or more of a signal strength of the plurality of data tags, a total scans per scanning period, a total number of scanners reading each of the plurality of data tags, and user information associated with a user positioning the plurality of data tags.

Example 50: The method of any one of examples 30 to 49, wherein the plurality of data tags includes a plurality of RFID tags.

Example 51: The method of any one of examples 30 to 50, wherein the plurality of data tags are each coupled to a medication stored within the medication station.

Example 52: The method of any one of examples 30 to 51, wherein the performance metric includes a performance score.

Example 53: The method of any one of examples 30 to 52, further including: associating the performance metric with a first medication station; comparing the performance metric of the first medication station with a second performance metric associated with a second medication station, the second performance metric generated based on scan data associated with a second scan of a second plurality of data tags positioned within the second medication station; and displaying, via a user interface, information representing the comparing.

Example 54: The method of any one of examples 30 to 53, further including: associating the performance metric with a first medical facility; comparing the performance metric of the first medical facility with a second performance metric associated with a second medical facility, the second performance metric generated based on scan data associated with a second scan of a second plurality of data tags positioned within a second medication station of the second medical facility; and displaying, via a user interface, information representing the comparing.

Example 55: The method of any one of examples 30 to 54, further including: displaying, via a user interface, the performance metric.

Example 56: A non-transitory computer-readable medium storing instructions, which when executed by at least one data processor, result in operations including: receiving, from a scanner of a medication station, scan data associated with a scan of a plurality of data tags positioned within the medication station; generating, based on the scan data, a performance metric indicating a performance of the plurality of data tags; and adjusting, based on the performance metric, a scan parameter of the scanner of the plurality of data tags and/or transmitting, based on the performance metric, an indication associated with the performance of the plurality of data tags.

Example 57: An apparatus including: means for receiving, from a scanner of a medication station, scan data associated with a scan of a plurality of data tags positioned within the medication station; means for generating, based on the scan data, a performance metric indicating a performance of the plurality of data tags; and means for adjusting, based on the performance metric, a scan parameter of the scanner of the plurality of data tags and/or transmitting, based on the performance metric, an indication associated with the performance of the plurality of data tags.

Example 58: The apparatus of example 57, further including: means for performing the method of any one of examples 30 to 55.

Example 59: A method, including: scanning, by a scanner of a medication station, a plurality of data tags coupled to a plurality of medications positioned within the medication station; generating, by the scanner and during the scanning, scan data associated with the scanning; transmitting, by the scanner, the scan data; and receiving, by the scanner, a control signal causing an adjustment of the scanning of the plurality of data tags by the scanner.

Example 60: A non-transitory computer-readable medium storing instructions, which when executed by at least one data processor, result in operations including: scanning, by a scanner of a medication station, a plurality of data tags coupled to a plurality of medications positioned within the medication station; generating, by the scanner and during the scanning, scan data associated with the scanning; transmitting, by the scanner, the scan data; and receiving, by the scanner, a control signal causing an adjustment of the scanning of the plurality of data tags by the scanner.

Example 61: An apparatus including: means for scanning, by a scanner of a medication station, a plurality of data tags coupled to a plurality of medications positioned within the medication station; means for generating, by the scanner and during the scanning, scan data associated with the scanning; means for transmitting, by the scanner, the scan data; and means for receiving, by the scanner, a control signal causing an adjustment of the scanning of the plurality of data tags by the scanner.

Example 62: A method, including: deep scanning a data tag coupled to a medication stored in a medication station, wherein the medication station includes a scanner configured to scan the data tag; generating, during the deep scanning, scan data associated with the scanning; generating, based on the scan data, a performance metric indicating a performance of the data tag; and adjusting, based on the performance metric, a scan parameter of the scanner of the data tag and/or transmitting, based on the performance metric, an indication associated with the performance of the data tag.

Example 63: A non-transitory computer-readable medium storing instructions, which when executed by at least one data processor, result in operations including: deep scanning a data tag coupled to a medication stored in a medication station, wherein the medication station includes a scanner configured to scan the data tag; generating, during the deep scanning, scan data associated with the scanning; generating, based on the scan data, a performance metric indicating a performance of the data tag; and adjusting, based on the performance metric, a scan parameter of the scanner of the data tag and/or transmitting, based on the performance metric, an indication associated with the performance of the data tag.

Example 64: An apparatus including: means for deep scanning a data tag coupled to a medication stored in a medication station, wherein the medication station includes a scanner configured to scan the data tag; means for generating, during the deep scanning, scan data associated with the scanning; means for generating, based on the scan data, a performance metric indicating a performance of the data tag; and means for adjusting, based on the performance metric, a scan parameter of the scanner of the data tag and/or transmitting, based on the performance metric, an indication associated with the performance of the data tag.

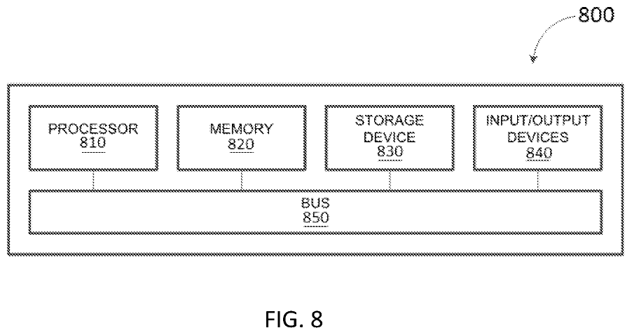

FIG. 8 depicts a block diagram illustrating a computing system 800 consistent with implementations of the current subject matter. Referring to FIGS. 1-7, the computing system 800 can be used to implement the adaptive wireless scanning management system 100, such as the analytics engine 110, and/or any components therein.

As shown in FIG. 8, the computing system 800 can include a processor 810, a memory 820, a storage device 830, and input/output device 840. The processor 810, the memory 820, the storage device 830, and the input/output device 840 can be interconnected via a system bus 850. The processor 810 is capable of processing instructions for execution within the computing system 800. Such executed instructions can implement one or more components of, for example, the analytics engine 110 and/or the adaptive wireless scanning management system 100. In some example implementations, the processor 810 can be a single-threaded processor. Alternatively, the processor 810 can be a multi-threaded processor. The processor 810 is capable of processing instructions stored in the memory 820 and/or on the storage device 830 to display graphical information for a user interface provided via the input/output device 840.

The memory 820 is a computer readable medium such as volatile or non-volatile that stores information within the computing system 800. The memory 820 can store data structures representing configuration object databases, for example. The storage device 830 is capable of providing persistent storage for the computing system 800. The storage device 830 can be a floppy disk device, a hard disk device, an optical disk device, a tape device, a solid-state device, and/or any other suitable persistent storage means. The input/output device 840 provides input/output operations for the computing system 800. In some example implementations, the input/output device 840 includes a keyboard and/or pointing device. In various implementations, the input/output device 840 includes a display unit for displaying graphical user interfaces.

According to some example implementations, the input/output device 840 can provide input/output operations for a network device. For example, the input/output device 840 can include Ethernet ports or other networking ports to communicate with one or more wired and/or wireless networks (e.g., a local area network (LAN), a wide area network (WAN), the Internet).

In some example implementations, the computing system 800 can be used to execute various interactive computer software applications that can be used for organization, analysis and/or storage of data in various formats. Alternatively, the computing system 800 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 840. The user interface can be generated and presented to a user by the computing system 800 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A system, comprising:
at least one data processor; and
at least one memory storing instructions which, when executed by the at least one data processor, result in operations comprising:
receiving, from a scanner of a medication station, scan data associated with a scan of a plurality of data tags positioned within the medication station;
generating, based on the scan data, a performance metric indicating a performance of the plurality of data tags; and
adjusting, based on the performance metric, a scan parameter of the scanner of the plurality of data tags and/or transmitting, based on the performance metric, an indication associated with the performance of the plurality of data tags.

2. The system of claim 1, wherein the scan parameter comprises a selection of the scanner from a plurality of scanners of the medication station to scan at least one data tag of the plurality of data tags.

3. The system of claim 1, wherein the scan parameter comprises one or more of a length of the scan of the plurality of data tags, a scanning path of the scanner during the scan of the plurality of data tags, a frequency of the scan of the plurality of data tags, and a scanning interval between consecutive scans of the plurality of data tags.

4. (canceled)

5. The system of claim 4, wherein the deep scan is performed to one or more of assess the performance of the plurality of data tags and improve the performance of the plurality of data tags.

6. The system of claim 4, wherein the adjusting comprises dynamically adjusting an inventory scan setting of an inventory scan of the plurality of data tags, the inventory scan determining an inventory of a plurality of medications within the medication station after the medication station is accessed, wherein each of the plurality of data tags are coupled to a corresponding medication of the plurality of medications.

7. The system of claim 1, wherein the indication indicates one or more of a failure of at least one data tag of the plurality of data tags, an unacceptable degradation of at least one data tag of the plurality of data tags, use of an unauthorized data tag, improper placement of at least one data tag of the plurality of data tags, and failure of the scanner.

8. The system of claim 1, wherein the operations further comprise:
comparing the performance metric to a prior performance metric and/or to a baseline performance metric; and
determining the performance metric does not correspond to the prior performance metric and/or the baseline performance metric.

9. The system of claim 8, wherein the operations further comprise: generating, based on the determination, a prediction of a failure of at least one data tag of the plurality of data tags; and wherein the indication comprises the prediction.

10. The system of claim 8, wherein the operations further comprise: detecting, based on the determination, use of an unauthorized data tag; and logging, based on the detection, the unauthorized data tag.

11. The system of claim 1, wherein the indication comprises: a recommendation for one or more of positioning of the plurality of data tags, performing a maintenance check of the plurality of data tags, and adjusting the scan parameter.

12. The system of claim 1, wherein the operations further comprise: determining the performance metric fails to correspond to a threshold metric.

13. The system of claim 1, wherein the operations further comprise:
receiving, from the scanner, updated scan data associated with an updated scan of the plurality of data tags, the updated scan occurring after the adjusting and/or the transmitting; and
generating, based on the updated scan data, an updated performance metric indicating an updated performance of the plurality of data tags.

14. The system of claim 13, wherein the operations further comprise: adjusting, based on the updated performance metric, an updated scan parameter of the updated scan of the plurality of data tags and/or transmitting, based on the updated performance metric, an updated indication associated with the performance of the plurality of data tags.

15. The system of claim 13, wherein the operations further comprise: requesting one or more of a barcode scan of the plurality of data tags, key access to the medication station, and a witness to view a user accessing the medication station.

16. (canceled)

17. (canceled)

18. (canceled)

19. (canceled)

20. The system of claim 1, wherein the plurality of scan data comprises one or more of a signal strength of the plurality of data tags, a total scans per scanning period, a total number of scanners reading each of the plurality of data tags, and user information associated with a user positioning the plurality of data tags.

21. (canceled)

22. The system of claim 1, wherein the plurality of data tags are each coupled to a medication stored within the medication station.

23. (canceled)

24. The system of claim 1, wherein the operations further comprise:
associating the performance metric with a first medication station;
comparing the performance metric of the first medication station with a second performance metric associated with a second medication station, the second performance metric generated based on scan data associated with a second scan of a second plurality of data tags positioned within the second medication station; and
displaying, via a user interface, information representing the comparing.

25. The system of claim 1, wherein the operations further comprise:
associating the performance metric with a first medical facility;
comparing the performance metric of the first medical facility with a second performance metric associated with a second medical facility, the second performance metric generated based on scan data associated with a second scan of a second plurality of data tags positioned within a second medication station of the second medical facility; and
displaying, via a user interface, information representing the comparing.

26. (canceled)

27. A system, comprising:
at least one data processor; and
at least one memory storing instructions which, when executed by the at least one data processor, result in operations comprising:
scanning, by a scanner of a medication station, a plurality of data tags coupled to a plurality of medications positioned within the medication station;
generating, by the scanner and during the scanning, scan data associated with the scanning;
transmitting, by the scanner, the scan data; and
receiving, by the scanner, a control signal causing an adjustment of the scanning of the plurality of data tags by the scanner.

28. A medication station, comprising:
at least one data processor; and
at least one memory storing instructions which, when executed by the at least one data processor, result in operations comprising:
deep scanning a data tag coupled to a medication stored in the medication station, wherein the medication station includes a scanner configured to scan the data tag;
generating, during the deep scanning, scan data associated with the scanning;
generating, based on the scan data, a performance metric indicating a performance of the data tag; and
adjusting, based on the performance metric, a scan parameter of the scanner of the data tag and/or transmitting, based on the performance metric, an indication associated with the performance of the data tag.

29-64. (canceled)
